(19) United States
(12) Patent Application Publication
(10) Pub. No.: US 2007/0128262 A1
(43) Pub. Date: Jun. 7, 2007

(54) Package containing adhesive patch and method of inhibiting drug migration

(76) Inventors: Satoshi Amano, Ibaraki (JP); Sachiko Honma, Ibaraki (JP); Tetsuro Tateishi, Ibaraki (JP); Naruhito Higo, Saga (JP)

Correspondence Address: NATH & ASSOCIATES, 112 South West Street, Alexandria, VA, 22314, US

(21) Appl. No.: 10/587,660

(22) PCT Filed: Jan. 31, 2005
(86) PCT No.: PCT/JP05/01348

§ 371 Date: Sep. 28, 2006

(30) Foreign Application Priority Data
Jan. 30, 2004 (JP) 2004-024470

(51) Int. Cl. A61K 9/70; A61K 31/138

(52) U.S. Cl. 424/449; 514/651

(57) ABSTRACT

The patch-containing pouch 100 of the present invention is a pouch 1 housing in its interior a patch 2 which has a pressure-sensitive adhesive layer 22 laminated on at least one side of a support 21 and has a release film 23 attached to the pressure-sensitive adhesive layer 22, wherein the pressure-sensitive adhesive layer 22 contains a drug represented by general formula (1) or a pharmaceutically acceptable salt thereof, and at least a portion of the inner surface 31a or 31b of the pouch 1 in contact with the patch 2 is made of polyacrylonitrile. 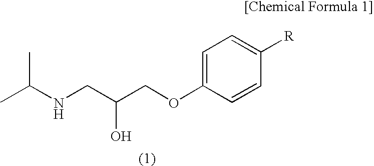 [where R represents 2-isopropoxyethoxymethyl, carbamoylmethyl or 2-methoxyethyl.]

### TECHNICAL FIELD

The present invention relates to a patch-containing pouch and to a method for inhibiting drug migration.

### BACKGROUND ART

Methods known for preventing adsorption and migration of an organic liquid component in a patch onto the inner surface of a packaging material include a method which employs a plastic material having a solubility parameter of 9 or greater for the inner surface of the packaging material (Patent document 1). Drugs such as bisoprolol are used as active ingredients in patches, and in such cases, cellonium pouches employing cellophane as the inner surface material are used. Patent document 1: Japanese Patent Application Laid-Open No. HEI 5-305108

### DISCLOSURE OF THE INVENTION

### Problem to be Solved by the Invention

However, the present inventors have noticed that significant migration often occurs depending on the components in such patches, even if the plastic material has a solubility parameter of 9 or greater. Particularly when the drug used is bisoprolol or a compound with a chemical skeletal structure similar thereto, it is not possible to prevent adhesion even when using a film made of an ethylene/vinyl alcohol copolymer or acrylonitrile/methyl acrylate copolymer with a solubility parameter of 9 or greater.

It is therefore an object of the present invention to provide a patch-containing pouch which is a pouch housing a patch containing bisoprolol or a compound with a chemical skeletal structure similar thereto, wherein the inner surface of the pouch is resistant to adhesion of drugs.

### Means for Solving the Problem

In order to achieve the object stated above, the patch-containing pouch of the present invention is a pouch housing in its interior a patch which has a pressure-sensitive adhesive layer laminated on at least one side of a support and has a release film attached to the pressure-sensitive adhesive layer, the pouch being characterized in that the pressure-sensitive adhesive layer contains a drug represented by general formula (1) below or a pharmaceutically acceptable salt thereof, and that at least a portion of the inner surface of the pouch in contact with the patch is made of polyacrylonitrile. 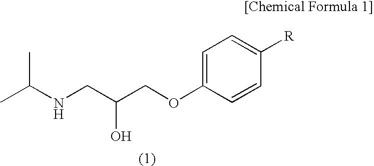
[where R represents 2-isopropoxyethoxymethyl, carbamoylmethyl or 2-methoxyethyl.]

A patch-containing pouch having such a construction can inhibit migration of a drug represented by general formula (1) (hereinafter referred to as a “drug (1)”). Specifically, a drug (1) has high migration properties and migrates onto a support and a release film, and after reaching the inner layer of the pouch, it spreads over the entire inner layer of the pouch; however, adhesion of the drug can be prevented if polyacrylonitrile is used for the inner surface. It is thereby possible to minimize migration of the drug.

The drug used may be a drug wherein R of general formula (1) is 2-isopropoxyethoxymethyl, carbamoylmethyl or 2-methoxyethyl. While these drugs selectively block β₁ receptors of the sympathetic nervous system and are useful as antihypertensive drugs exhibiting hypotensive effects, their use has been limited because of significant drug migration with conventional pouches. However, by using the pouch according to the present invention, it is possible to inhibit drug migration even with patches containing the drugs mentioned above, thus allowing a high drug content to be maintained in the patch even after prolonged storage.

The pressure-sensitive adhesive layer of the patch preferably contains at least one type of pressure-sensitive adhesive selected from the group consisting of acrylic-based pressure-sensitive adhesives containing a polymer including a (meth)acrylic acid ester as a monomer unit, block copolymer-based pressure-sensitive adhesives containing a styrene-based block copolymer, and pressure-sensitive adhesives comprising the aforementioned acrylic-based pressure-sensitive adhesive and the aforementioned block copolymer-based pressure-sensitive adhesive. Such a pressure-sensitive adhesive is useful as a base because it can hold the aforementioned drug and permit increased transdermal absorption of the drug.

Preferably, the pouch is constructed of a multilayer film and the layer of the multilayer film forming the inner surface of the pouch is made of polyacrylonitrile. When the inner surface film is made of a material with high drug permeability, interlayer peeling of the multilayer film may occur during storage of the patch, but when polyacrylonitrile is used for the inner surface layer, adhesion of a drug (1) itself is inhibited and interlayer peeling problems are prevented. It is particularly preferred for the entire inner surface layer to be made of polyacrylonitrile, and this makes it possible to efficiently inhibit drug adhesion. In other words, drug adhesion can be prevented even if the patch containing the drug shifts in the interior of the pouch after it has been housed in the pouch.

Also, the layer of the multilayer film forming the outer surface of the pouch is preferably made of polyethylene terephthalate. By using polyethylene terephthalate as the outer surface, it is possible to physically protect the inner surface layer while inhibiting degeneration, i.e. corrosion and the like, of the outer surface of the pouch.

In addition, the patch-containing pouch is also preferably provided with a layer made of aluminum between the layer of the multilayer film forming the inner surface and the layer of the multilayer film forming the outer surface. By providing the patch-containing pouch with an aluminum layer, it is possible not only to inhibit migration of the drug contained in the patch, but also to prevent volatilization of the volatile components of the formulation, out of the pouch and infiltration of moisture into the pouch, thereby improving the gas barrier property.

The present invention further provides a method for inhibiting drug migration whereby migration of a drug onto the inner surface of a pouch housing a patch provided with a pressure-sensitive adhesive layer containing the drug is inhibited, wherein the drug is a drug represented by general formula (1) or a pharmaceutically acceptable salt thereof, and at least a portion of the inner surface is a surface made of polyacrylonitrile.

According to this method, it is possible to prevent adhesion of the drug onto the inner surface of the pouch, and to maintain the drug content of the patch from the time of manufacture until the time of use of the patch.

### EFFECTS OF THE INVENTION

The patch-containing pouch according to the present invention can inhibit migration of a drug such as bisoprolol contained in a patch. In particular, by housing a patch in the interior of the pouch after manufacture of the patch until the time of use, it is possible to maintain the drug content in the patch while also ensuring migration of a sufficient dose of the drug into skin or the like at the time of use.

### BRIEF DESCRIPTION OF THE DRAWINGS

FIG. 1 is a cross-sectional view of a patch-containing pouch according to a first embodiment.

FIG. 2 is a cross-sectional view of a patch-containing pouch according to a second embodiment.

FIG. 3 is a cross-sectional view of a patch according to a first mode.

FIG. 4 is a cross-sectional view of a patch according to a second mode.

FIG. 5 is a cross-sectional view of a patch according to a third mode.

### EXPLANATION OF REFERENCE NUMERALS

100: First patch-containing pouch; 110: second patch-containing pouch; 1: pouch; 2: patch; 4: desiccant; 10a, 10b: multilayer films; 11a, 11b: inner layers; 12a, 12b: interlayers; 13a, 13b: outer layers; 21: support; 22: pressure-sensitive adhesive layer; 23: release film; 31a, 31b: inner surfaces.

### BEST MODES FOR CARRYING OUT THE INVENTION

Preferred embodiments of the present invention will now be explained in detail, with reference to the accompanying drawings.

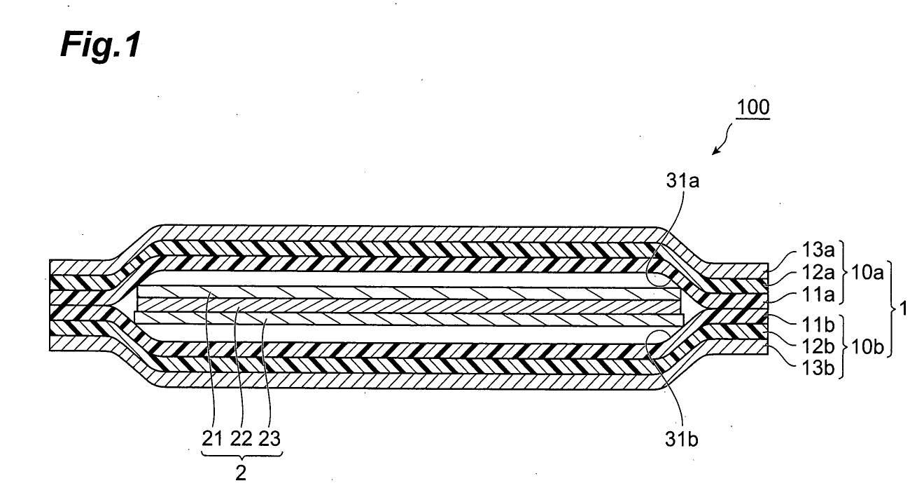

FIG. 1 is a cross-sectional view of a patch-containing pouch according to a first embodiment. The patch-containing pouch 100 shown in FIG. 1 houses a patch 2 in the interior of a pouch 1.

The pouch 1 is constructed of a multilayer film 10a obtained by laminating an inner layer 11a, an interlayer 12a and an outer layer 13 a in this order, and a multilayer film 10b obtained by laminating an inner layer 11b, an interlayer 12b and an outer layer 13b in this order, and the opposing inner layers 11a and 11b are bonded at the edges of the multilayer films 10a and 10b.

The patch 2 has a support 21, a pressure-sensitive adhesive layer 22 and a release film 23 laminated in this order, with a drug (1) contained in the pressure-sensitive adhesive layer 22. The patch 2 is in contact with the inner surfaces 31a and 31b of the pouch 1, i.e. the surfaces of the inner layers 11a and 11b, and the inner layers 11a and 11b in contact with the patch 2 are made of polyacrylonitrile. The interlayers 12a and 12b are made of aluminum, and the outer layers 13a and 13b are made of polyethylene terephthalate.

When the patch-containing pouch 100 constructed in this manner is stored for prolonged periods, the drug (1) contained in the pressure-sensitive adhesive layer 22 of the patch 2 gradually bleeds out from the base composing the pressure-sensitive adhesive layer 22, and migrates along the support 21 or the release film 23. Migration of the drug (1) onto the surfaces of the inner layers 11a and 11b often continues until the entire surfaces of the inner layers 31a and 31b become covered with the drug (1). This gradually reduces the amount of the drug (1) in the pressure-sensitive adhesive layer 22 and fouls the surface or inner surfaces 31a and 31b of the patch 2 with the drug (1). Thus, even when the pouch 1 is sealed to store the patch 2, a significant amount of the drug (1) bleeds from the patch 2 in the interior of the pouch 1, such that the stored patch 2 becomes no longer suitable for use.

With the patch-containing pouch 100 of the first embodiment, however, the inner layers 11a and 11b are made of polyacrylonitrile so that migration of the drug (1) onto the surfaces of the inner layers 11a and 11b is inhibited and the amount of bleeding of the drug (1) from the pressure-sensitive adhesive layer 22 is reduced. Hence, there is no reduction of the drug (1) in the patch 2, allowing use even after prolonged storage.

Since the interlayers 12a and 12b are made of aluminum in the patch-containing pouch 100 of the first embodiment, not only is migration of the drug contained in the patch inhibited, but it is also possible to prevent volatilization of the volatile components out of the pouch and infiltration of moisture into the pouch, thereby improving the gas barrier property. Moreover, since the outer layers 13a and 13b are made of polyethylene terephthalate, the interlayers 12a and 12b are protected, and prevented from wearing, so that the thicknesses of the interlayers 12a and 12b may be maximally reduced.

Gaps between the patch 2 and the inner portion of the patch-containing pouch 100 may also be eliminated in order to more notably exhibit the effect described above. Yet even if gaps are present on the inner portion of the pouch 1 allowing movement of the patch 2, migration of the drug (1) is inhibited when the entirety of the inner layers 11a and 11b is made of polyacrylonitrile.

While the pouch 1 is constructed of a multilayer film in the patch-containing pouch 100 of the first embodiment described above, alternatively the pouch 1 may instead be constructed of a single-layer polyacrylonitrile film. Also, another resin may be used instead of polyethylene terephthalate for the outer layers 13a and 13b. As other resins to be used for the outer layers 13a and 13b, there may be mentioned polyethylene, polypropylene, nylon, cellophane, polyvinylidene chloride and ethylene-vinyl alcohol copolymer.

Also, although both of the inner layers 11a and 11b are made of polyacrylonitrile in the patch-containing pouch 100 of the first embodiment, optionally only one of the inner layers 11a and 11b may be made of polyacrylonitrile. Alternatively, polyacrylonitrile may be used for only portions of the inner layer 11a or 11b, particularly at least the portions which are in contact with the patch 2.

From the standpoint of gas permeability and manageability of the pouch, the thickness of the multilayer film 10a or 10b is preferably 20-100 μm. A thickness of less than 20 μm will lead to insufficient strength and may invite damage, thus impairing the airtightness, while a thickness of greater than 100 μm will impair the flexibility of the film, possibly leading to poor manageability.

The multilayer films 10a and 10b of the pouch 1 may be produced by sticking together the inner layers 11a and 11b, the interlayers 12a and 12b and the outer layers 13a and 13b by heat lamination, a publicly known method, or with an adhesive.

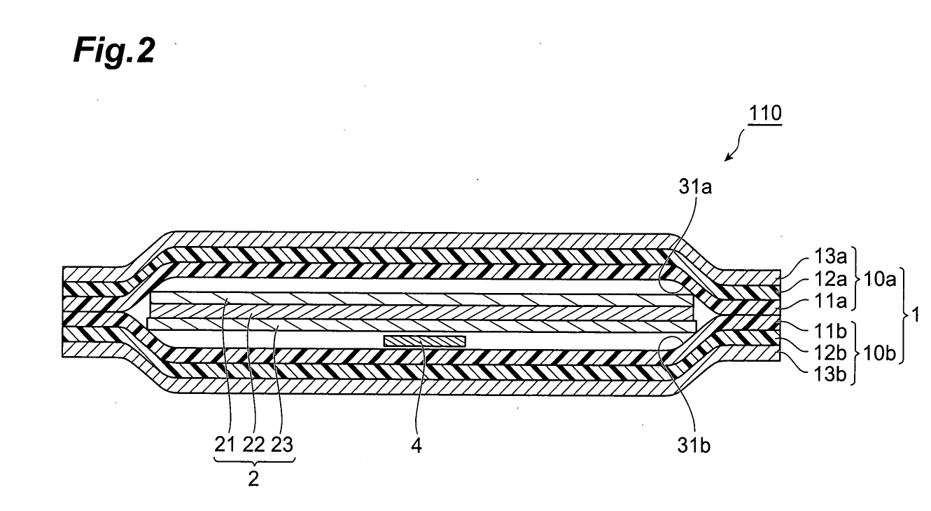

FIG. 2 is a cross-sectional view of a patch-containing pouch according to a second embodiment. This patch-containing pouch 110 has the same construction as the first embodiment, except that a desiccant 4 is situated in the interior gaps of the pouch 1.

Because the second embodiment includes the desiccant 4, it can maintain a high drug content. The drug (1) used for the present invention is known to be prone to migration and readily hydrolyzed. Thus, by including the desiccant 4, the drug content of the patch 2 can be maintained even during prolonged storage.

There are no particular restrictions on the desiccant 4, and as examples there may be mentioned viscous minerals such as silica gel, synthetic or natural zeolite, and montmorillonite.

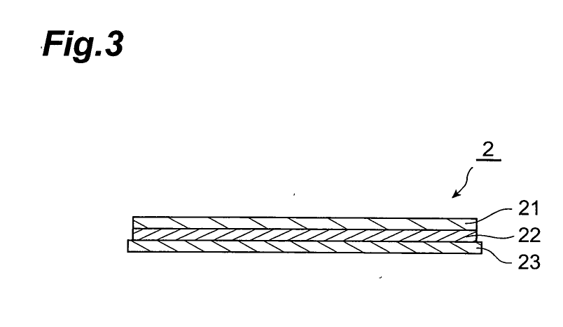

FIGS. 3 to 5 are cross-sectional views of first to third modes of a patch housed in the interior of a pouch 1.

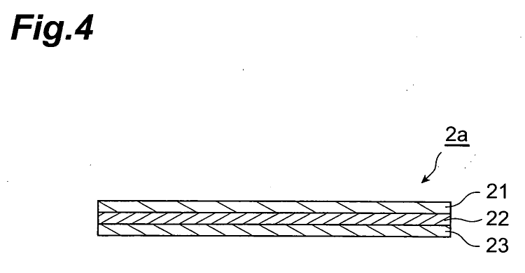

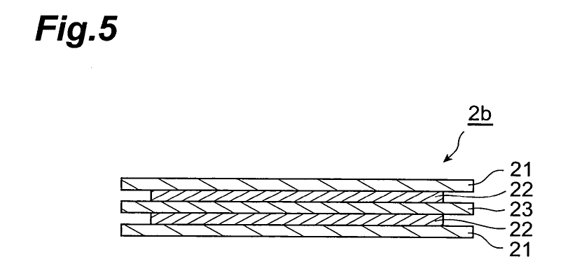

The patch 2 of the first mode shown in FIG. 3 corresponds to the patch 2 shown in FIGS. 1 and 2, having a pressure-sensitive adhesive layer 22 with a smaller area than a release film 23 and with an area equivalent to a support 21, sandwiched between the support 21 and the release film 23. The patch 2a of the second mode shown in FIG. 4 has a support 21, a pressure-sensitive adhesive layer 22 and a release film 23 with equivalent areas laminated in this order. The patch 2b of the third mode shown in FIG. 5 has a pressure-sensitive adhesive layer 22 and a support 21 laminated in this order on the side of the release film 23 on which a pressure-sensitive adhesive layer 22 is not formed in FIG. 3. In FIGS. 3 to 5, the release film 23 is laminated in a releasable manner on the pressure-sensitive adhesive layer 22.

According to the present invention, migration of the drug (1) can still be adequately inhibited when the patch 2a, which has the pressure-sensitive adhesive layer 22 exposed at the edges as shown in FIG. 4 and which is more prone to migration onto the surfaces of the inner layers 11a and 11b, is housed in the interior of the pouch 1.

The drug contained in the pressure-sensitive adhesive layer 22 is a drug represented by general formula (1) or a pharmaceutically acceptable salt thereof. Drugs represented by general formula (1), in general, selectively block β1 receptors of the sympathetic nervous system and have a hypotensive action. Drugs wherein R of general formula (1) is 2-isopropoxyethoxymethyl are particularly suitable for use because they are indicated for amelioration of essential hypertension, angina and arrhythmias. The term “pharmaceutically acceptable salt thereof” refers to a salt of a drug of general formula (1) above which exhibits the same pharmacological activity.

The content of the drug in the pressure-sensitive adhesive layer 22 is preferably 1-50 wt % and more preferably 5-20 wt % based on the total weight of the pressure-sensitive adhesive layer 22. If the drug content is less than 1 wt %, release of the drug from the pressure-sensitive adhesive layer 22 will be hampered, making it difficult to administer a suitable dose of the drug during use. On the other hand, if the content is greater than 50 wt %, the drug will not be held in the pressure-sensitive adhesive layer 22, resulting in inferior pressure-sensitive adhesive properties.

The pressure-sensitive adhesive layer 22 is made of a pressure-sensitive adhesive composition containing the drug (1), and if necessary, it may also contain a tackifier, softener and the like. As pressure-sensitive adhesives in the pressure-sensitive adhesive composition, there may be mentioned acrylic-based pressure-sensitive adhesives containing a polymer including a (meth)acrylic acid ester as a monomer unit, block copolymer-based pressure-sensitive adhesives containing a styrene-based block copolymer, and pressure-sensitive adhesives comprising the aforementioned acrylic-based pressure-sensitive adhesive and the aforementioned block copolymer-based pressure-sensitive adhesive, because of their excellent pressure-sensitive adhesive properties and excellent drug release properties. There are no particular restrictions on the acrylic-based pressure-sensitive adhesive so long as it is obtained by copolymerization with at least one (meth)acrylic acid (ester), which may typically be acrylic acid, 2-ethylhexyl acrylate, methyl acrylate, butyl acrylate, hydroxyethyl acrylate, 2-ethylhexyl methacrylate or the like, and as examples there may be mentioned 2-ethylhexyl acrylate-vinyl acetate copolymer, 2-ethylhexyl acrylate-vinyl acetate-acrylic acid copolymer, 2-ethylhexyl acrylate-vinyl acetate-hydroxyethyl acrylate copolymer, 2-ethylhexyl acrylate-vinyl acetate-hydroxyethyl acrylate-acrylic acid copolymer and 2-ethylhexyl acrylate-2-ethylhexyl methacrylate-dodecyl methacrylate copolymer, among which 2-ethylhexyl acrylate-vinyl acetate copolymer and 2-ethylhexyl acrylate-vinyl acetate-acrylic acid copolymer are particularly preferred. As styrene-based block copolymers there may be mentioned styrene-isoprene-styrene block copolymer (SIS), styrene-butadiene-styrene block copolymer (SBS), styrene-ethylene-butylene-styrene block copolymer (SEBS) and styrene-ethylene-propylene-styrene block copolymer (SEPS), among which styrene-isoprene-styrene block copolymer (SIS) is particularly preferred. The pressure-sensitive adhesive is more preferably a mixture of SIS and 2-ethylhexyl acrylate-vinyl acetate-acrylic acid copolymer.

As softeners there may be mentioned petroleum-based oils (for example, paraffinic processed oils, naphthenic processed oils and aromatic processed oils), squalane, squalene, vegetable oils (for example, almond oil, olive oil, camellia oil, castor oil, tall oil and peanut oil), olefinic acids, silicone oil, dibasic acid esters (for example, dibutyl phthalate and dioctyl phthalate), liquid rubbers (for example, polybutene and liquid isoprene rubber), liquid fatty acid esters (for example, isopropyl myristate, hexyl laurate, diethyl sebacate and isopropyl sebacate), diethylene glycol, polyethylene glycol, glycol salicylate, propylene glycol, dipropylene glycol, triacetin, triethyl citrate and crotamiton. Particularly preferred among these are liquid paraffin, isopropyl myristate and diethyl sebacate because they can impart a suitable degree of adhesion onto skin. These softeners may be used as single types alone, or two or more thereof may be used in combination.

As tackifiers there may be mentioned alicyclic saturated hydrocarbon resins, rosin derivatives (for example, rosin, rosin glycerin esters, hydrogenated rosin, hydrogenated rosin glycerin esters and rosin pentaerythritol esters), terpene resins, petroleum resins and maleic acid resins. Particularly preferred among these are alicyclic saturated hydrocarbon resins and hydrogenated rosin glycerin esters. These tackifiers may be used as single types alone, or two or more thereof may be used in combination.

The support 21 is preferably a support which satisfactorily maintains the migration properties of the drug while providing superior flexibility, and as examples there may be mentioned films, nonwoven fabrics, woven fabrics and knitted fabrics, which are made of polyethylene terephthalate, polyethylene, polypropylene, polyvinyl chloride, ethylene-vinyl acetate copolymer, polyurethane and the like. Preferred among these are polyethylene terephthalate and ethylene-vinyl acetate copolymer.

The release film 23 used may be for example a resin film made of polyethylene terephthalate or polypropylene, or release-treated paper, and a silicone-treated polyethylene terephthalate film is particularly preferred.

There are no particular restrictions on the method of producing the patch 2, and for example, the drug, pressure-sensitive adhesive and softener may be heat melted, and coated onto the release film 23 or support 21 to form the pressure-sensitive adhesive layer 22, and this may be attached to the support 21 or release film 23 to obtain the patch 2. Alternatively, the drug, pressure-sensitive adhesive and softener may be dissolved in a solvent such as toluene, hexane, heptane or ethyl acetate, and spread onto the release film 23 or support 21, and the solvent removed by drying to form the pressure-sensitive adhesive layer 22, and this may be attached to the support 21 or release film 23 to obtain the patch 2.

The patch-containing pouches 100 and 110 are produced by housing the patch 2 or the patch 2 and desiccant 4 in the pouch 1, and bonding together the edges of the multilayer films 10a and 10b. Thus, the content stability of the drug contained in the patch 2 will also depend on the environment of the interior gaps of the pouch 1. The relative humidity in the gaps is preferably kept at no greater than 25%. Conditions may be adjusted during the manufacturing process, or by adding the desiccant 4 or the like, to be included in the aforementioned conditions.

The present invention also provides a method for inhibiting drug migration which allows migration of a drug from the patch 2 to the pouch 1 to be inhibited. By using a pouch 1 with polyacrylonitrile as the inner surface, it is possible to inhibit migration of a drug of general formula (1) contained in the patch 2, from the patch 2 to the pouch 1. According to this method, it is possible to prevent adhesion of the drug onto the inner surface of the pouch 1 and to maintain the drug content of the patch from the time of manufacture until the time of use of the patch.

### EXAMPLES

The present invention will now be explained in greater detail by examples and comparative examples, with the understanding that the present invention is in no way limited to the examples.

### Example 1

(Fabrication of Pouch A)

A multilayer film A (85 mm length, 79 mm width, 40 μm thickness) having a polyacrylonitrile film (PAN, 20 μm thickness), aluminum foil (Al, 7 μm thickness) and polyester film (PET, 12 μm thickness) laminated in this order was prepared.

Next, multilayer films A were situated with their polyacrylonitrile layers facing each other, and three of the edges were bonded by heat sealing and air-cooled to obtain a pouch A having one edge open for loading of a patch.

(Fabrication of Patch A)

TABLE 1

| Formulation A | Addition (wt %) |
|---|---|
| Duro-Tak 87-2194 | 26.5 |
| SIS | 10.0 |
| ARKON P-100 | 40.5 |
| Liquid paraffin | 5.0 |
| Diethyl sebacate | 8.0 |
| Bisoprolol | 10.0 |

Of the components of formulation A shown in Table 1 above, bisoprolol, liquid paraffin and diethyl sebacate were placed in a container and thoroughly mixed together. The mixture was then combined with a solution obtained by dissolving SIS, Duro-Tak 87-2194 (acrylic-based pressure-sensitive adhesive solution, product of National Starch & Chemical Co., Ltd.) and ARKON P-100 (alicyclic saturated hydrocarbon resin, product of Arakawa Chemical Industries, Ltd.) in toluene, to prepare a coating solution. The obtained coating solution was applied onto a silicone-treated polyethylene terephthalate release film, the solvent was removed by drying to form a pressure-sensitive adhesive layer, and this was attached to a polyethylene terephthalate support to obtain a desired patch. The obtained patch was cut to 10 cm² (square). The thickness of the pressure-sensitive adhesive layer of the patch was 100 μm.

(Fabrication of Patch-Containing Pouch A)

The patch A was loaded into the pouch A, and the open edge was bonded by heat sealing to obtain a sealed patch-containing pouch A.

### Example 2

A pouch A and patch A were fabricated according to Example 1.

(Fabrication of Patch-Containing Pouch B)

The patch A and a desiccant (silica gel, by Sud-Chemie) were loaded into the pouch A and the edge was bonded by heat sealing to obtain a sealed patch-containing pouch B.

### Comparative Example 1

A commercially available cellonium packaging material (a four-layer packaging material, with the inner three layers consisting of a cellophane film, a polyethylene film and an aluminum foil in this order from the innermost side, and the outer layer consisting of a polyester film) (product of Toppan Printing Co., Ltd.) was used instead of the pouch A.

(Fabrication of Patch-Containing Pouch C)

The patch A was loaded into the cellonium packaging material, and the edge was bonded by heat sealing to obtain a sealed patch-containing pouch C.

### Comparative Example 2

A commercially available cellonium packaging material (product of Toppan Printing Co., Ltd.) was used instead of the pouch A.

(Fabrication of Patch-Containing Pouch D)

The patch A and a desiccant (silica gel, by Sud-Chemie) were loaded into the cellonium packaging material and the edge was bonded by heat sealing to obtain a sealed patch-containing pouch D.

(Evaluation Method)

(Stability Test)

The drug contents (n0) of the patch-containing pouches A-D immediately after fabrication in Examples 1 and 2 and Comparative Examples 1 and 2 were each measured. They were then stored in a thermo-hygrostat at 60° C. and the drug contents (n1) were again measured after one month. The drug content ratio (R) was then calculated from the relationship represented by equation (2) below.
R=(n1/n0)×100 (2)

The results are shown in Table 2.

TABLE 2

| | Drug content ratio (%) |
|---|---|
| Example 1 | 96.6 |
| Example 2 | 98.0 |
| Comparative Example 1 | 95.1 |
| Comparative Example 2 | 96.0 |

As clearly seen in Table 2, the patch-containing pouches of the present invention obtained in Examples 1 and 2 had high drug content ratios even after storage in a thermo-hygrostat at 60° C. for 1 month, and the pouch obtained in Example 2, which included a desiccant, was able to maintain a particularly high drug content ratio. In contrast, the pouches obtained in Comparative Examples 1 and 2, which had no polyacrylonitrile contained in the inner surfaces, exhibited notably reduced drug content ratios after storage. This confirmed that the patch-containing pouches of the present invention are highly effective for inhibiting migration of drugs contained in patches and maintaining drug contents.

1-6. (canceled)

7. A patch-containing pouch housing in its interior a patch which has a pressure-sensitive adhesive layer laminated on at least one side of a support and has a release film attached to said pressure-sensitive adhesive layer,
wherein said pressure-sensitive adhesive layer contains a drug represented by general formula (1) below or a pharmaceutically acceptable salt thereof, and at least a portion of the inner surface of said pouch in contact with said patch is made of polyacrylonitrile. 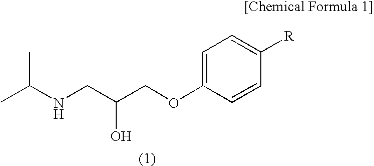
[where R represents 2-isopropoxyethoxymethyl, carbamoylmethyl or 2-methoxyethyl.]

8. The patch-containing pouch according to claim 7, wherein said pressure-sensitive adhesive layer contains at least one type of pressure-sensitive adhesive selected from the group consisting of acrylic-based pressure-sensitive adhesives containing a polymer including a (meth)acrylic acid ester as a monomer unit, block copolymer-based pressure-sensitive adhesives containing a styrene-based block copolymer, and pressure-sensitive adhesives comprising said acrylic-based pressure-sensitive adhesive and said block copolymer-based pressure-sensitive adhesive.

9. The patch-containing pouch according to claim 7, wherein said pouch is constructed of a multilayer film, and the layer of said multilayer film forming the inner surface of said pouch is made of polyacrylonitrile.

10. The patch-containing pouch according to claim 8, wherein said pouch is constructed of a multilayer film, and the layer of said multilayer film forming the inner surface of said pouch is made of polyacrylonitrile.

11. The patch-containing pouch according to claim 9, wherein the layer of said multilayer film forming the outer surface of said pouch is made of polyethylene terephthalate.

12. The patch-containing pouch according to claim 10, wherein the layer of said multilayer film forming the outer surface of said pouch is made of polyethylene terephthalate.

13. The patch-containing pouch according to claim 11, which is provided with a layer made of aluminum between the layer of said multilayer film forming the inner surface and the layer of said multilayer film forming the outer surface.

14. The patch-containing pouch according to claim 12, which is provided with a layer made of aluminum between the layer of said multilayer film forming the inner surface and the layer of said multilayer film forming the outer surface.

15. A method for inhibiting drug migration whereby migration of a drug onto the inner surface of a pouch housing a patch provided with a pressure-sensitive adhesive layer containing said drug is inhibited,
wherein
said drug is a drug represented by general formula (1) below or a pharmaceutically acceptable salt thereof, and
at least a portion of said inner surface is a surface made of polyacrylonitrile. 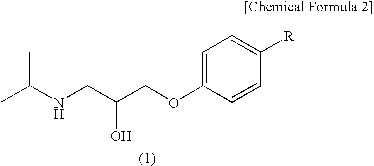
[where R represents 2-isopropoxyethoxymethyl, carbamoylmethyl or 2-methoxyethyl.]
